(19) United States
(12) Patent Application Publication
(10) Pub. No.: US 2018/0161469 A1
(43) Pub. Date: Jun. 14, 2018

(54) Toilet Deodorizer

(71) Applicant: NINGBO JIANGBEI OCEAN STAR TRADING CO., LTD., Ningbo (CN)

(72) Inventor: Haiying Jiang, Ningbo (CN)

(21) Appl. No.: 15/374,274

(22) Filed: Dec. 9, 2016

(51) Int. Cl. A61L 9/01

(52) U.S. Cl. CPC A61L 9/01

(57) ABSTRACT

The present invention discloses a toilet deodorizer, comprising the following components in percentage by weight: 0.5-7% of fragrance, 0.7-15% of a solubilizer, less than or equal to 0.1% of a preservative, 0.5-8% of a deodorant, 0.1-0.7% of amino acid, 8-15% of propylene glycol, less than or equal to 1% of a toner and the balance of water. The toilet deodorizer is natural in component, harmless, efficient and environment-friendly, convenient to use and rapid in deodorization.

### FIELD OF THE INVENTION

The present invention belongs to the technical field of daily chemicals, and in particular relates to a toilet deodorizer.

### BACKGROUND OF THE INVENTION

After being used for a certain time, toilets in families, hospitals, hotels and the like will have odor. Chemical analysis on the odor shows that the odor mainly contains substances such as an ammonia gas, hydrogen sulfide, amine and sulfuric acid, and the odor in toilets can make people unpleasant.

To eliminate and cover the odor, the most commonly-used method is to adopt an adsorption deodorizer or a covering deodorizer. The adsorption deodorizer is a deodorization method in which stink molecules are adsorbed into a porous substance by using a substance with an excellent adsorption property by virtue of the Van der Waals' force of molecules. The deodorizer is large in specific surface area and large in air capacity, the stink degree of the air can be decreased through adsorption, and thus the purpose of deodorization can be achieved. When the covering deodorizer is used, the stink is covered by substances such as natural aromatic oil and perfume. The covering deodorizer is mainly used in environments with heavy stink which is hard to remove or relatively troublesome to remove, and the stink can be alleviated by mixing multiple scented gases according to a ratio. The adsorption deodorizer has the defects that the odor cannot be thoroughly adsorbed and can be only eliminated after a long time of adsorption. The perfume covering mode can only paralyze the smell of people, but the odor substances cannot be fundamentally removed.

### SUMMARY OF THE INVENTION

To solve the problem of the prior art, the present invention provides a toilet deodorizer.

To achieve the purpose, the technical solution of the present invention is that the toilet deodorizer comprises the following components in percentage by weight: 0.5-7% of fragrance, 0.7-15% of a solubilizer, less than or equal to 0.1% of a preservative, 0.5-8% of a deodorant, 0.1-0.7% of amino acid, 8-15% of propylene glycol, less than or equal to 1% of a toner and the balance of water.

The fragrance is capable of refreshing air, odor which is hard to remove or odor in an environment in which odor is troublesome to remove can be covered by using perfume. The solubilizer is used for solubilizing the fragrance, and the ratio of the solubilizer needs to be changed along with the change of the ratio of the fragrance. The preservative is used for enhancing the preservative property of a final product. The deodorant can have irreversible reaction with molecules (H₂S, NH₃) in eliminated odor to generate harmless and odorless substances. The amino acid is mainly used for preventing precipitation of a product. The propylene glycol is mainly used for preventing freezing of the product, and is beneficial to emission and diffusion of the fragrance. The toner is mainly used for adjusting the color of the product, and thus the product can be different from other similar products. As a solvent, the water is used for mixing the substances in the ratio.

The preparation method of the toilet deodorizer comprises the following steps: firstly, mixing the fragrance with the solubilizer, and uniformly stirring; adding the deodorant, the amino acid and the propylene glycol in sequence, and uniformly stirring; finally, adding the water and the preservative, and uniformly stirring; adding the toner to adjust the color of the product according to demands finally. By adopting the technical solution, the deodorant in the toiler deodorizer can have irreversible reaction with molecules (H₂S, NH₃) in eliminated odor to generate harmless and odorless substances, so that the odor generated when people go to a toilet can be fundamentally removed. The toilet deodorizer has natural ingredients and is harmless, efficient and environment-friendly, convenient to use, rapid in deodorization, low in cost and simple in preparation process.

### DETAILED DESCRIPTION OF THE EMBODIMENTS

The technical solution of the present disclosure is further described with the following embodiment:

The toilet deodorizer comprises the following components in percentage by weight: 0.5-7% of fragrance, 0.7-15% of a solubilizer, less than or equal to 0.1% of a preservative, 0.5-8% of a deodorant, 0.1-0.7% of amino acid, 8-15% of propylene glycol, less than or equal to 1% of a toner and the balance of water.

The solubilizer can be one of hydrogenated castor oil polyoxyethylene ether, polyethylene glycol mono-octylphenol ether, sorbitan monooleate, polyoxyethylene sorbitan monooleat and polyoxyethylene sorbitan monostearate. Zinc ricinoleate is preferred as the deodorant. The amino acid is L-arginine. The preservative is Kathon. The deodorant is a mixture consisting of 30% of zinc ricinoleate, 5% of 1-hydroxyethylidene-1,1-diphosphonic acid, 15% of hydrogenated castor oil polyoxyethylene ether and 50% of water. The chemical formula of zinc ricinoleate is as follows:

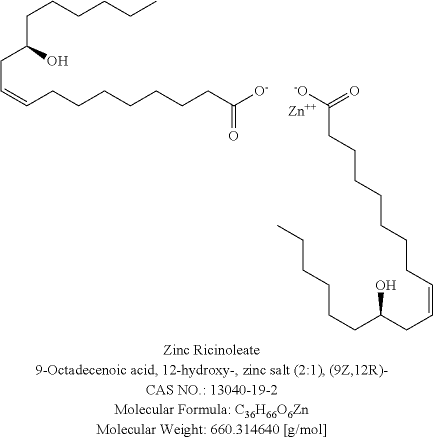

When being used, the deodorant functions in a main mode that zinc atoms in the deodorant and nitrogen atoms and sulfur atoms in odor form very strong chemical bonds, and thus the smell generated when people go to toilets can be fundamentally removed. As a substitute of a deodorant, the CM0007 deodorant produced by the Shanghai Xiaojianjing Fragrance Co., Ltd. can be adopted as the deodorant, and the content of the effective substance soyethyl morpholinium ethosulfate in the CM0007 deodorant is 70%.

The fragrance and the deodorant play a leading role in the toilet deodorizer. The fragrance degree of the product is mainly reflected by the change of the fragrance. The deodorization effect of the product is taken into play through the deodorant, and to relatively sufficiently describe the solution, the addition amounts of the preservative, the propylene glycol and the toner in the following embodiment are not changed.

### Embodiment 1: 0.5% of fragrance

| | | Ratio 1 | Ratio 2 | Ratio 3 | Ratio 4 | Ratio 5 | |
|---|---|---|---|---|---|---|---|
| | | (poor | (relatively poor | (medium | (relatively good | (good | |
| Component | | deodorization | deodorization | deodorization | deodorization | deodorization | |
| name | Chemical name | property) | property) | property) | property) | property) | Function |
| Fragrance | Fragrance | 0.5% | 0.5% | 0.5% | 0.5% | 0.5% | Faint scent |
| Solubilizer | Hydrogenated | 0.7% | 0.7% | 0.7% | 0.7% | 0.7% | Solubilizing |
| | castor oil | | | | | | fragrance |
| | polyoxyethylene | | | | | | |
| | ether | | | | | | |
| Preservative | Kathon | 0.1% | 0.1% | 0.1% | 0.1% | 0.1% | Preservation |
| Deodorant | Zinc | 0.5% | 1% | 3% | 5% | 8% | Deodorization |
| | ricinoleate | | | | | | |
| Amino acid | L-arginine | 0.1% | 0.2% | 0.3% | 0.5% | 0.7% | Precipitation |
| | | | | | | | prevention |
| Propylene | Propylene | 12% | 12% | 12% | 12% | 12% | Prevent |
| glycol | glycol | | | | | | freezing, |
| | | | | | | | promote |
| | | | | | | | fragrance |
| | | | | | | | emission |
| Toner | Toner | 0.1% | 0.1% | 0.1% | 0.1% | 0.1% | Adjust color |
| Water | Water | 86.0% | 85.4% | 83.3% | 81.1% | 77.9% | Solvent |

### Embodiment 2: 1% of fragrance

| | | Ratio 1 | Ratio 2 | Ratio 3 | Ratio 4 | Ratio 5 | |
|---|---|---|---|---|---|---|---|
| | | (poor | (relatively poor | (medium | (relatively good | (good | |
| Component | | deodorization | deodorization | deodorization | deodorization | deodorization | |
| name | Chemical name | property) | property) | property) | property) | property) | Function |
| Fragrance | Fragrance | 1% | 1% | 1% | 1% | 1% | Faint scent |
| Solubilizer | Hydrogenated | 2% | 2% | 2% | 2% | 2% | Solubilizing |
| | castor oil | | | | | | fragrance |
| | polyoxyethylene | | | | | | |
| | ether | | | | | | |
| Preservative | Kathon | 0.1% | 0.1% | 0.1% | 0.1% | 0.1% | Preservation |
| Deodorant | Zinc | 0.5% | 1% | 3% | 5% | 8% | Deodorization |
| | ricinoleate | | | | | | |
| Amino acid | L-arginine | 0.1% | 0.2% | 0.3% | 0.5% | 0.7% | Precipitation |
| | | | | | | | prevention |
| Propylene | Propylene | 12% | 12% | 12% | 12% | 12% | Prevent |
| glycol | glycol | | | | | | freezing, |
| | | | | | | | promote |
| | | | | | | | fragrance |
| | | | | | | | emission |
| Toner | Toner | 0.1% | 0.1% | 0.1% | 0.1% | 0.1% | Adjust color |
| Water | Water | 84.2% | 83.6% | 81.5% | 79.3% | 76.1% | Solvent |

### Embodiment 3: 3% of fragrance

| | | Ratio 1 | Ratio 2 | Ratio 3 | Ratio 4 | Ratio 5 | |
|---|---|---|---|---|---|---|---|
| | | (poor | (relatively poor | (medium | (relatively good | (good | |
| Component | | deodorization | deodorization | deodorization | deodorization | deodorization | |
| name | Chemical name | property) | property) | property) | property) | property) | Function |
| Fragrance | Fragrance | 3% | 3% | 3% | 3% | 3% | Faint scent |
| Solubilizer | Hydrogenated | 6% | 6% | 6% | 6% | 6% | Solubilizing |
| | castor oil | | | | | | fragrance |
| | polyoxyethylene | | | | | | |
| | ether | | | | | | |
| Preservative | Kathon | 0.1% | 0.1% | 0.1% | 0.1% | 0.1% | Preservation |
| Deodorant | Zinc | 0.5% | 1% | 3% | 5% | 8% | Deodorization |
| | ricinoleate | | | | | | |
| Amino acid | L-arginine | 0.1% | 0.2% | 0.3% | 0.5% | 0.7% | Precipitation |
| | | | | | | | prevention |
| Propylene | Propylene | 12% | 12% | 12% | 12% | 12% | Prevent |
| glycol | glycol | | | | | | freezing, |
| | | | | | | | promote |
| | | | | | | | fragrance |
| | | | | | | | emission |
| Toner | Toner | 0.1% | 0.1% | 0.1% | 0.1% | 0.1% | Adjust color |
| Water | Water | 78.2% | 77.6% | 75.5% | 73.3% | 70.1% | Solvent |

### Embodiment 4: 5% of fragrance

| | | Ratio 1 | Ratio 2 | Ratio 3 | Ratio 4 | Ratio 5 | |
|---|---|---|---|---|---|---|---|
| | | (poor | (relatively poor | (medium | (relatively good | (good | |
| Component | | deodorization | deodorization | deodorization | deodorization | deodorization | |
| name | Chemical name | property) | property) | property) | property) | property) | Function |
| Fragrance | Fragrance | 5% | 5% | 5% | 5% | 5% | Faint scent |
| Solubilizer | Hydrogenated | 10% | 10% | 10% | 10% | 10% | Solubilizing |
| | castor oil | | | | | | fragrance |
| | polyoxyethylene | | | | | | |
| | ether | | | | | | |
| Preservative | Kathon | 0.1% | 0.1% | 0.1% | 0.1% | 0.1% | Preservation |
| Deodorant | Zinc | 0.5% | 1% | 3% | 5% | 8% | Deodorization |
| | ricinoleate | | | | | | |
| Amino acid | L-arginine | 0.1% | 0.2% | 0.3% | 0.5% | 0.7% | Precipitation |
| | | | | | | | prevention |
| Propylene | Propylene | 12% | 12% | 12% | 12% | 12% | Prevent |
| glycol | glycol | | | | | | freezing, |
| | | | | | | | promote |
| | | | | | | | fragrance |
| | | | | | | | emission |
| Toner | Toner | 0.1% | 0.1% | 0.1% | 0.1% | 0.1% | Adjust color |
| Water | Water | 72.2% | 71.6% | 69.5% | 67.3% | 64.1% | Solvent |

### Embodiment 5: 7% of fragrance

| | | Ratio 1 | Ratio 2 | Ratio 3 | Ratio 4 | Ratio 5 | |
|---|---|---|---|---|---|---|---|
| | | (poor | (relatively poor | (medium | (relatively good | (good | |
| Component | | deodorization | deodorization | deodorization | deodorization | deodorization | |
| name | Chemical name | property) | property) | property) | property) | property) | Function |
| Fragrance | Fragrance | 7% | 7% | 7% | 7% | 7% | Faint scent |
| Solubilizer | Hydrogenated | 15% | 15% | 15% | 15% | 15% | Solubilizing |
| | castor oil | | | | | | fragrance |
| | polyoxyethylene | | | | | | |
| | ether | | | | | | |
| Preservative | Kathon | 0.1% | 0.1% | 0.1% | 0.1% | 0.1% | Preservation |
| Deodorant | Zinc | 0.5% | 1% | 3% | 5% | 8% | Deodorization |
| | ricinoleate | | | | | | |
| Amino acid | L-arginine | 0.1% | 0.2% | 0.3% | 0.5% | 0.7% | Precipitation |
| | | | | | | | prevention |
| Propylene | Propylene | 12% | 12% | 12% | 12% | 12% | Prevent |
| glycol | glycol | | | | | | freezing, |
| | | | | | | | promote |
| | | | | | | | fragrance |
| | | | | | | | emission |
| Toner | Toner | 0.1% | 0.1% | 0.1% | 0.1% | 0.1% | Adjust color |
| Water | Water | 65.2% | 64.6% | 62.5% | 60.3% | 57.1% | Solvent |

See the deodorization effect of the toilet deodorizer in the following table:

| | | Concentration | Concentration | |
|---|---|---|---|---|
| | | before the | after the | |
| Content of | Odor | deodorizer is | deodorizer is | Deodorization |
| deodorant | ingredient | used (ppm) | used (ppm) | rate |
| 0.5% | H₂S | 95 | 45 | 52.63% |
| 0.5% | NH₃ | 190 | 100 | 47.36% |
| 1% | H₂S | 95 | 30 | 68.42% |
| 1% | NH₃ | 190 | 70 | 63.16% |
| 3% | H₂S | 95 | 20 | 78.95% |
| 3% | NH₃ | 190 | 40 | 78.95% |
| 5% | H₂S | 95 | 10 | 89.47% |
| 5% | NH₃ | 190 | 15 | 92.1% |
| 8% | H₂S | 95 | 5 | 94.73% |
| 8% | NH₃ | 190 | 5 | 97.37% |

The present invention is not limited by the optimal embodiment above, products of other modes can be obtained by any person with the enlightenment of the present invention, but no matter how the shape or structure is changed, technical solutions identical to or similar as the present invention are included by the protection scope of the present invention.

1. A toilet deodorizer, comprising the following components in percentage by weight: 0.5-7% of fragrance, 0.7-15% of a solubilizer, less than or equal to 0.1% of a preservative, 0.5-8% of a deodorant, 0.1-0.7% of amino acid, 8-15% of propylene glycol, less than or equal to 1% of a toner and the balance of water.

2. The toilet deodorizer according to claim 1, wherein the solubilizer can be one of hydrogenated castor oil polyoxyethylene ether, polyethylene glycol mono-octylphenol ether, sorbitan monooleate, polyoxyethylene sorbitan monooleat and polyoxyethylene sorbitan monostearate; the amino acid is L-arginine; and the preservative is Kathon.

3. The toilet deodorizer according to claim 2, wherein the deodorant is a mixture consisting of 30% of zinc ricinoleate, 5% of 1-hydroxyethylidene-1,1-diphosphonic acid, 15% of hydrogenated castor oil polyoxyethylene ether and 50% of water.

4. The toilet deodorizer according to claim 3, comprising 0.5-7% of fragrance, 0.7-15% of solubilizer, 0.1% of preservative, 0.5% of deodorant, 0.1% of amino acid, 12% of propylene glycol, 1% of toner and the balance of water.

5. The toilet deodorizer according to claim 3, comprising 0.5-7% of fragrance, 0.7-15% of solubilizer, 0.1% of preservative, 1% of deodorant, 0.2% of amino acid, 12% of propylene glycol, 1% of toner and the balance of water.

6. The toilet deodorizer according to claim 3, comprising 0.5-7% of fragrance, 0.7-15% of solubilizer, 0.1% of preservative, 3% of deodorant, 0.3% of amino acid, 12% of propylene glycol, 1% of toner and the balance of water.

7. The toilet deodorizer according to claim 3, comprising 0.5-7% of fragrance, 0.7-15% of solubilizer, 0.1% of preservative, 5% of deodorant, 0.5% of amino acid, 12% of propylene glycol, 1% of toner and the balance of water.

8. The toilet deodorizer according to claim 3, comprising 0.5-7% of fragrance, 0.7-15% of solubilizer, 0.1% of preservative, 8% of deodorant, 0.7% of amino acid, 12% of propylene glycol, 1% of toner and the balance of water.
